(19) United States
(12) Plant Patent Application Publication
(10) Pub. No.: US 2012/0278960 P1
(43) Pub. Date: Nov. 1, 2012

(54) Osteospermum plant named 'SAKOST8201'

(50) ;

(75) Inventor: Niels G. Kristensen, Silkeborg (DK)

(73) Assignee: Sakata Seed Corporation, Yokohama (JP)

(21) Appl. No.: 13/068,016

(22) Filed: Apr. 29, 2011

(51) Int. Cl. A01H 5/00

(52) U.S. Cl. PLT/360

(57) ABSTRACT

A new Osteospermum plant named ‘SAKOST8201’ particularly distinguished by having white with purple colored flowers and a semi-erect plant habit, is disclosed.

### GENUS AND SPECIES

Osteospermum hybrida

### VARIETY DENOMINATION

‘SAKOST8201’

### BACKGROUND OF THE NEW PLANT

The present invention comprises a new and distinct variety of Osteospermum hybrida designated ‘SAKOST8201’. ‘SAKOST8201’ originated from a cross conducted in May 2006 in Aabyhoej, Denmark between hybrid Osteospermum breeding lines ‘205102’ and ‘205103’. The male parent, ‘205103’, has a bluish flower color and a compact plant habit. The female parent, ‘205102’, has a soft bluish purple flower color, well branching and a relatively vigorous plant growth habit.

The seeds were sown and 200 plants were grown in pots for evaluation. Out of 200 F₁ lines, the breeder selected a single plant having white with purple colored flowers and a semi-erect plant habit for further evaluation and asexual propagation.

In the Spring of 2007, plants were evaluated in 16.0 centimeter pots and in an open field. Osteospermum ‘SAKOST8201’ has been found to retain its distinctive characteristics through successive asexual propagations via vegetative cuttings.

‘SAKOST8201’ has not been made publicly available for more than one year prior to the filing of this application.

### SUMMARY OF THE INVENTION

The following are the most outstanding and distinguishing characteristics of this new variety when grown under normal horticultural practices in a greenhouse in Salinas, Calif.

1. White with purple colored flowers; and

2. Semi-erect plant habit.

### DESCRIPTION OF THE PHOTOGRAPHS

This new Osteospermum plant is illustrated by the accompanying photographs which show the overall plant habit including form, foliage, and flowers. The photographs are of a plant grown four months from transplant date and five months from stick date into 4-inch pots from rooted vegetative cuttings in Salinas, Calif. under greenhouse conditions. The colors shown are as true as can be reasonably obtained by conventional photographic procedures.

FIG. 1 shows overall plant habit of the plant grown in a pot.

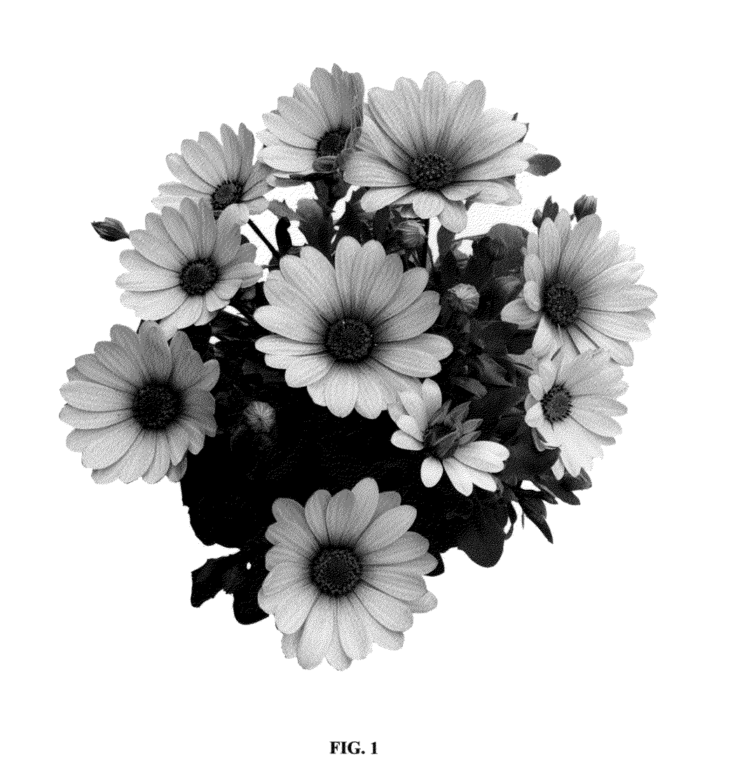

FIG. 2 shows the mature inflorescence of the plant.

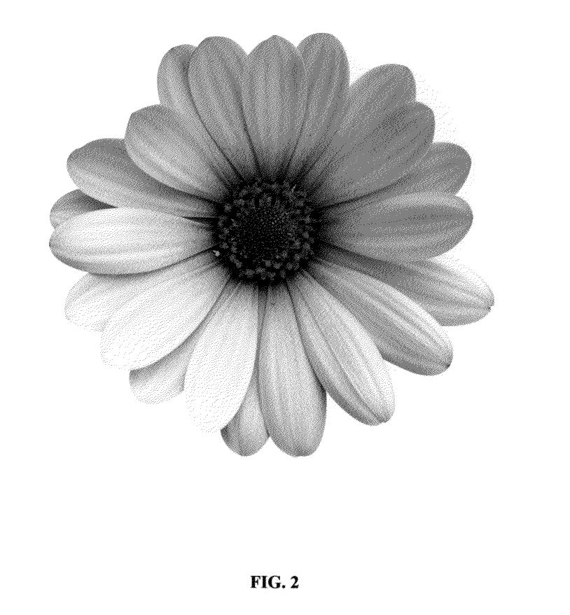

FIG. 3 shows three flowers with leaves.

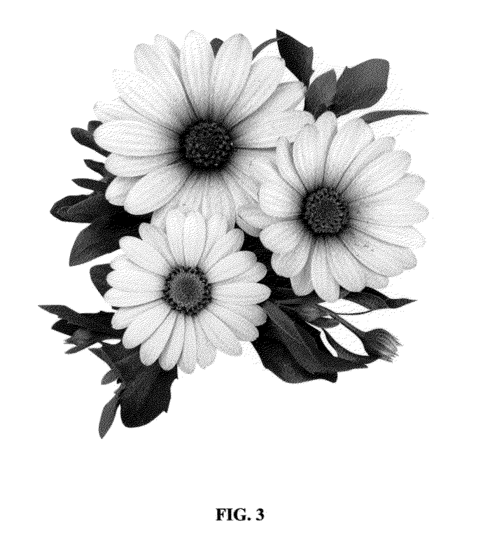

### DESCRIPTION OF THE NEW VARIETY

The following detailed descriptions set forth the distinctive characteristics of ‘SAKOST8201’. The data which define these characteristics were collected from asexual reproductions carried out in Salinas, Calif. Data were collected on plants grown approximately four months from transplant date and five months from stick date into 4-inch pots under greenhouse conditions in Salinas, Calif. Color references are to the RHS Colour Chart of The Royal Horticultural Society of London (R.H.S.), 4^{th} edition.

### DESCRIPTION OF THE NEW PLANT

Classification:
   Family.—Compositae.
   Botanical.—Osteospermum hybrida.
Parentage:
   Female parent.—Osteospermum line ‘205102’.
   Male parent.—Osteospermum line ‘205103’.
Growth:
Time to produce a rooted cutting: Vegetative cuttings will colonize a 2.5 centimeter diameter by 2.5 centimeter tall greenhouse tray cell with peat-based plant media in approximately four weeks. Cuttings are dipped in a normal dilution (1:9) of Dip ‘N Grow root inducing solution in water. The trays are misted hourly during rooting.
Environmental conditions for plant growth: Rooted cuttings are transplanted to pots with a 16 centimeter diameter, one plant per pot. Peat-based growing media is used. The pots are watered using a 150 ppm to 200 ppm fertilizer solution using 18-8-18 fertilizer. The soil is allowed to dry between watering. During the first few weeks after transplanting, the plants should have evening temperatures around 15° C. to 18° C. for good root growth. When plants reach 7.5 centimeters to 10.0 centimeters in height they are pinched back to 5 to 6 leaves to promote branching. Spring and summer daytime high temperatures in Salinas, Calif., where the data was collected, range from 16° C. to 25° C.
Time to bloom from propagation: Approximately four weeks when rooted vegetative cuttings are transferred to a 5 inch diameter pot. Flowering season is all year in the United States. Vernalization is not required to induce flowering.
Plant description:
   Habit.—Semi-erect.
   Life cycle.—Perennial.
   Form.—Semi-erect.
   Height (from soil line to first node).—1.0 cm.
   Height (from soil line to top of foliage).—33.0 cm to 34.0 cm.
   Width.—47.0 cm to 48.0 cm.
Stems:
   General.—Multiple, circular cross-section.
   Stem length (total).—8.0 cm to 10.0 cm.
   Diameter.—0.2 cm.
   Internode length.—1.5 cm.
   Color.—Green RHS 144A (Yellow-green).
   Anthocyanin color.—Slight, RHS 187A (Greyed-purple).
   Pubescence.—Slight. Color: RHS N155A (White) Shape: Short, hair-like fibers.
Branches:
   General.—Circular cross-section.
   Number of main branches.—1.
   Total number of branches.—8.
   Length.—11.0 cm to 12.0 cm, approximately 1.0 cm from soil line to first node, 1.0 cm between first and second nodes.
   Diameter.—0.4 cm.
   Internode length.—2.5 cm.
   Color.—RHS 144A (Yellow-green).
   Anthocyanin color.—Slight, RHS 187A (Greyed-purple).
   Pubescence.—Glabrous.
Leaves:
   Arrangement.—Alternate.
   Shape.—Oblanceolate.
   Apex.—Acute.
   Base.—Attenuate.
   Margin.—Serrate.
   Venation.—Pinnate.
   Surface.—Dull.
   Surface pubescence.—Very slight, mainly around edge.
   Pubescence color.—RHS N155A (White).
   Attachment.—Decurrant.
   Length.—7.0 cm.
   Width.—3.0 cm.
   Thickness.—Less than 0.1 cm.
   Color.—Upper surface: RHS 137A (Green) Lower surface: RHS 137B (Green).
   Venation color.—Upper surface: RHS 144B (Yellow-green) Lower surface: RHS 144C (Yellow-green).
Inflorescence:
   Number per plant.—42.
   Type.—Capitulum (head); disk florets are perfect and ray florets are carpellate.
   Flowering habit.—Determinate.
   Lastingness of flowers.—3 to 4 days.
   Fragrance.—Absent.
   Seed production.—Absent.
   Diameter.—7.0 cm.
   Depth.—1.0 cm.
   Petal number.—20 to 26 per inflorescence.
   Disc diameter.—1.3 cm.
   Disc floret number.—60 to 80 per inflorescence.
   Peduncle length.—5.0 cm from inflorescence to first node.
   Peduncle diameter.—0.2 cm.
   Peduncle color.—RHS 144A (Yellow-green).
   Peduncle texture.—Dull, slight pubescence.
   Phyllaries.—Arrangement: 22 per inflorescence, free, arranged symmetrically Length: 1.0 cm Width: 0.1 cm Apex: Acute Margin: Entire Shape: Linear, acute Color: Both surfaces: RHS 144A (Yellow-green).
Ray florets (Ligules):
   Corolla.—1 petal per ray floret; only the outer row of florets are the ray florets.
   Number of ray florets per inflorescence.—20 to 26.
   Petal length.—3.3 cm.
   Petal width.—0.9 cm.
   Petal shape.—Lanceolate.
   Petal apex.—Acute.
   Petal margin.—Entire.
   Petal color.—Upper surface: RHS 155D (White) with RHS 72B (Red-purple) stripes and at base Lower surface: RHS 155D (White) with stripes of RHS 77A, B and C (Red-purple).
   Petal pubescence.—Glabrous.
Disc florets:
   Bud color.—RHS 94B (Violet-blue).
   Shape.—Tubular.
   Apex.—Pointed.
   Surface.—Dull, pubescent.
   Size.—Length: 0.6 cm Width: 0.1 cm.
Reproductive organs:
   Gynoecium.—Location: Ray florets and disc florets Ovary: Superior Pistil form: 1 style with 2 stigma branches Pistil length: 0.5 cm Stigma color: RHS 187A (Greyed-purple) Style color: RHS 155A (White)
   Androecium.—Location: Disc florets only Stamens: 5 fused into a single tube Anther color: RHS 187A (Greyed-purple) Pollen color: RHS 17A (Yellow-orange) Filament color: RHS 155A (White).
Disease and insect resistance: ‘SAKOST8201’ is very disease resistant, however, plants are susceptible to aphids, thrips, whiteflies, and worms.

### COMPARISON WITH PARENTAL LINES AND KNOWN VARIETIES

‘SAKOST8201’ is distinguished from its parents as described in Table 1. Color references are to The Royal Horticultural Society Colour Chart, 4^{th} Edition.

TABLE 1
Comparison with Parental Lines

| | | Male Parent | Female Parent |
|---|---|---|---|
| Characteristic | ‘SAKOST8201’ | ‘205103’ | ‘205102’ |
| Ray floret (ligule) | RHS 155D (White) | Bluish | Bluish Purple |
| color, upper surface | with RHS 72B (Red- | | |
| | purple) stripes and | | |
| | at base | | |
| Plant growth habit | Semi-erect | Compact | Vigorous |

‘SAKOST8201’ is most similar to the Osteospermum plant named ‘Alczawhir’. However, there are differences as described in Table 2. Color references are to The Royal Horticultural Society Colour Chart, 4^{th} Edition.

TABLE 2
Comparison with Similar Variety

| Characteristic | ‘SAKOST8201’ | ‘Akzawhir’ |
|---|---|---|
| Ray floret (ligule) color, | RHS 155D (White) with | White with Purple |
| upper surface upper | RHS 72B (Red-purple) | ring around discs |
| surface | stripes and at base | |
| Branching | Very good | Good |
| Plant growth habit | Medium | Long |

I claim:

1. A new and distinct variety of Osteospermum plant named ‘SAKOST8201’ as shown and described herein.
